(19) United States
(12) Patent Application Publication
(10) Pub. No.: US 2007/0104672 A1
(43) Pub. Date: May 10, 2007

(54) Cosmetic composition comprising a cation, a solid fatty substance and a sorbitan ester, and cosmetic treatment process

(76) Inventors: Sandrine Decoster, Saint Gratien (FR); Patricia Mezure, Bougival (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/589,215

(22) Filed: Oct. 30, 2006

Related U.S. Application Data
(60) Provisional application No. 60/736,301, filed on Nov. 15, 2005.

(30) Foreign Application Priority Data
Oct. 28, 2005 (FR) 05 53285

(51) Int. Cl. A61K 8/46; A61K 8/42; A61K 8/37

(52) U.S. Cl. 424/70.28; 424/70.31

(57) ABSTRACT

The present disclosure relates to a cosmetic composition comprising, in a cosmetically acceptable aqueous medium, at least one cationic surfactant, at least one oxyethylenated sorbitan ester and at least one non-silicone solid fatty substance, in an oxyethylenated sorbitan ester/fatty substance weight ratio ranging from 0.2 to 50. The cosmetic compositions as disclosed herein have an improved conditioning effect, for instance on hair sheen.

This application claims benefit of U.S. Provisional Application No. 60/736,301, filed Nov. 15, 2005, the contents of which are incorporated herein by reference. This application also claims benefit of priority under 35 U.S.C. §119 to French Patent Application No. FR 05 53285, filed Oct. 28, 2005, the contents of which are also incorporated herein by reference.

The present disclosure relates to a cosmetic composition, for example a hair conditioner, comprising at least one cationic surfactant, at least one oxyethylenated sorbitan ester and at least one non-silicone solid fatty substance in a particular ratio, and to a cosmetic process for treating keratin materials, such as the hair.

It is well known that hair that has been sensitized (i.e., damaged and/or embrittled) to varying degrees under the action of atmospheric agents or under the action of mechanical or chemical treatments, such as dyeing, bleaching and/or permanent-waving, is often difficult to disentangle and to style, and may lack softness.

Cosmetic compositions comprising cationic surfactants have already been proposed for treating keratin materials, such as the hair.

However, such compositions may have drawbacks such as rinseability problems, stability problems, difficulties in distributing them over the keratin materials and/or also insufficient cosmetic properties.

It has also been recommended to use cationic polymers, cationic silicones or cationic surfactants in compositions for washing or caring for keratin materials such as the hair, to facilitate the disentangling of the hair and/or to give it softness and suppleness. The use of cationic polymers or cations for this purpose, however, may have various drawbacks. On account of their high affinity for the hair, some of these polymers may become deposited in a substantial amount during repeated use, and may lead to undesirable effects such as an unpleasant, laden feel, stiffening of the hair, and/or adhesion between the fibers that affects styling.

Furthermore, the care used for very sensitized hair may still be insufficient to treat the ends, which are usually very damaged.

In summary, it is found that the current conditioning cosmetic compositions are not always entirely satisfactory. Thus, it is sought to obtain cosmetic compositions that have very good cosmetic properties, for instance on very sensitized hair, and which furthermore have good skin tolerance; for example, the hair conditioning compositions of the prior art may cause discomfort reactions on the scalp and it would be desirable to avoid this.

International Patent Application WO 2004/093835 describes compositions containing cationic surfactants, fatty substances and oxyethylenated sorbitan esters. However, the concentrations of sorbitan esters used and the ester/fatty substance ratios are very low.

The present inventors have now discovered that the combination of a cationic surfactant, at least one oxyethylenated sorbitan ester and at least one non-silicone solid fatty substance in a particular ratio allows one or more of these drawbacks to be overcome.

Hair treated with the composition disclosed herein, such as sensitized hair, may be smooth, may disentangle easily, may be shiny, supple and individualized, and/or may have a soft feel with no feeling of residues. The treated hair may have a natural, unladen appearance. The smoothness may be uniform from the roots to the ends. The ends may show less splitting. These effects are remanent over time.

The present inventors have discovered that the addition of a particular weakly oxyethylenated sorbitan ester makes it possible, surprisingly, to reduce the discomfort reactions (itching, redness, etc.), for example on the scalp, of compositions containing surfactants liable to cause reactions of this type when they are used alone.

Thus, according to the present disclosure, novel cosmetic compositions are now proposed, comprising, in a cosmetically acceptable aqueous medium, at least one cationic surfactant, at least one ester of sorbitan and of a saturated or unsaturated, linear or branched C₈-C₃₀ fatty acid with a number of moles of ethylene oxide of less than or equal to 20, and at least one non-silicone solid fatty substance, wherein the at least one oxyethylenated sorbitan ester and the at least one non-silicone solid fatty substance are present in a weight ratio ranging from 0.2 to 50.

Another aspect of the present disclosure is a cosmetic process for treating keratin materials, such as the hair, using the above-mentioned composition.

Another aspect of the present disclosure is the use of the composition as a hair conditioner.

Other subjects, characteristics, aspects and advantages of the present disclosure will emerge even more clearly on reading the description and the various examples that follow.

According to the present disclosure, the term “sensitized hair” is generally understood to mean hair that has undergone external physical attack (by light, heat, waves, etc.), mechanical attack (by repeated blow-drying, combing or brushing, etc.) and/or chemical attack (by oxidation dyeing, bleaching, permanent-waving, relaxing, etc.). For instance, in at least one embodiment, the compositions according to the present disclosure are effective on hair sensitized by chemical attack.

As disclosed herein the term “at least one” will be generally understood as meaning “one or more”, i.e. one, two, three or more.

The term “cosmetically acceptable medium” means a medium that is compatible with any keratin material, such as the skin, the hair, the nails, the eyelashes, the eyebrows or the lips and any other area of the body and of the face.

For purposes of the present disclosure, the term “non-silicone fatty substance” means any oily organic substance not comprising any silicone atoms in its elemental structure, comprising at least one carbon-based chain containing at least 10 carbon atoms, and whose solubility in water at 25° C. (1 atm) is less than 0.1% by weight.

For instance, the solid fatty substances may have a melting point of greater than or equal to 35° C. and/or have a viscosity at a temperature of 40° C. and at a shear rate of 1 s⁻¹ of greater than or equal to 1 Pa-s.

According to at least one embodiment of the present disclosure, the at least one solid fatty substance is non-polymeric, i.e., it does not comprise any repeated monomer units other than alkylene oxide units.

The compositions according to the present disclosure are non-washing (non-detergent) compositions, and, in at least one embodiment, comprise less than 3% by weight, such as less than 1% by weight, of detergent surfactants, such as anionic surfactants, relative to the total weight of the composition. In a further embodiment, the composition does not contain any detergent surfactants.

According to at least one embodiment of the present disclosure, the oxyethylenated sorbitan ester/solid fatty substance weight ratio ranges from 0.25 to 10, for example from 0.3 to 5, such as from 0.4 to 2 or from 0.5 to 1.75.

The fatty acids of the sorbitan esters of a C₈-C₃₀ fatty acid, with a number of moles of ethylene oxide of less than or equal to 20, may, in at least one embodiment, comprise from 8 to 24 carbon atoms and further, for example, from 8 to 18 carbon atoms. In at least one embodiment, the fatty acids may be chosen from lauric acid, palmitic acid, oleic acid and stearic acid, for instance from lauric acid and stearic acid. In a further embodiment, the fatty acid is lauric acid.

Monoesters of a C₈-C₂₄ fatty acid and of oxyethylenated sorbitan may be used in at least one embodiment. For instance, the number of moles of ethylene oxide may be less than 10, such as from 3 to 8, mol of ethylene oxide, and in a further embodiment may be equal to 4 mol.

As disclosed herein, sorbitan esters may include but are not limited to sorbitan monolaurate oxyethylenated with 4 mol of ethylene oxide (4 EO) or polysorbate 21, sorbitan monostearate oxyethylenated with 4 mol of ethylene oxide (4 EO) or polysorbate 61, and sorbitan monooleate oxyethylenated with 5 mol of ethylene oxide (5 EO) or polysorbate 81.

According to at least one embodiment of the present disclosure, polysorbate 21 may be used, and is sold under the name TWEEN 21 by the company Uniqema.

According to the present disclosure, the oxyethylenated sorbitan ester may be present in the cosmetic composition in an amount ranging from 0.4% to 10%, for example ranging from 0.4% to 8% by weight relative to the total weight of the composition.

According to at least one embodiment, when the compositions are used in a process without final rinsing, the oxyethylenated sorbitan ester may be present in the cosmetic composition in an amount ranging from 0.4% to 5%, for instance from 2% to 6% by weight relative to the total weight of the composition.

According to another embodiment, when the compositions are used in a process with final rinsing, the oxyethylenated sorbitan ester may be present in the cosmetic composition in an amount ranging from 0.4% to 8%, for instance from 0.4% to 2% by weight relative to the total weight of the composition.

The composition according to the present disclosure comprises at least one cationic surfactant that is well known per se, such as optionally polyoxyalkylenated primary, secondary or tertiary fatty amine salts and quaternary ammonium salts, and mixtures thereof.

According to the present disclosure, the cationic surfactants are non-polymeric, i.e.,. they do not comprise any repeating monomer units. In at least one embodiment, the cationic surfactants according to the present disclosure may contain one or two functions capable of forming cationic charges.

Fatty amines that may be mentioned include but are not limited to alkylamidoamines, for instance (C₈-C₃₀)alkylamidodi(C₁-C₆)alkylamines, such as stearamidopropyldimethylamine (MACKINE 301 sold by Maclntyre).

Examples of quaternary ammonium salts that may be mentioned include but are not limited to:

those having the formula (V) below:

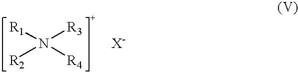

in which R₁ to R₄, which may be identical or different, are chosen from linear or branched aliphatic radicals containing from 1 to 30 carbon atoms and from aromatic radicals such as aryl or alkylaryl. The aliphatic radicals may comprise heteroatoms such as, for example, oxygen, nitrogen, sulfur and halogens. The aliphatic radicals may be chosen, for example, from (C₁-C₃₀)alkyl, alkoxy, (C₂-C₆) polyoxyalkylene, alkylamide, (C₁₂-C₂₂)alkylamido(C₂-C₆)alkyl, (C₁₂-C₂₂)alkyl acetate and hydroxyalkyl radicals, containing from 1 to 30 carbon atoms; X⁻ is an anion chosen from halides, phosphates, acetates, lactates, (C₂-C₆)alkyl sulfates and alkylsulfonates or alkylarylsulfonates;

quaternary ammonium salts of imidazoline, for instance those of formula (VI) below: 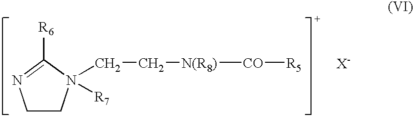
in which
R₅ is an alkenyl or alkyl radical containing from 8 to 30 carbon atoms, for example fatty acid derivatives of tallow or of coconut,
R₆ is chosen from a hydrogen atom, a C₁-C₄ alkyl radical and an alkenyl or alkyl radical having from 8 to 30 carbon atoms,
R₇ is a C₁-C₄ alkyl radical,
R₈ is chosen from a hydrogen atom and a C₁-C₄ alkyl radical, and
X⁻ is an anion chosen from halides, phosphates, acetates, lactates, alkyl sulfates, alkylsulfonates and alkylarylsulfonates. According to at least one embodiment, R₅ and R₆ denote a mixture of alkenyl or alkyl radicals containing from 12 to 21 carbon atoms, for example fatty acid derivatives of tallow, R₇ is methyl, and R₈ is hydrogen. Such a product is, for example, Quaternium-27 (CTFA 2002), Quaternium-87 (CTFA 2002) or Quaternium-83 (CTFA 2002), which are sold under the name “Varisoft®” W575PG by the company Goldschmidt,

diquaternary ammonium salts of formula (VII):

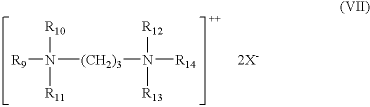

in which
R₉ is an aliphatic radical containing from 16 to 30 carbon atoms,
R₁₀, R₁₁, R₁₂, R₁₃ and R₁₄, which may be identical or different, are chosen from hydrogen and an alkyl radical containing from 1 to 4 carbon atoms, and
X⁻ is an anion chosen from halides, acetates, phosphates, nitrates, ethyl sulfates and methyl sulfates.
Such diquaternary ammonium salts comprise, for example, propanetallowdiammonium dichloride;

quaternary ammonium salts comprising at least one ester function, such as those of formula (VIII) below:

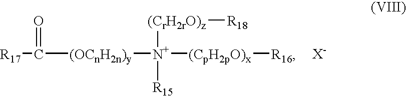

in which:

R₁₅ is chosen from C₁l-C₆ alkyl radicals and C₁-C6 hydroxyalkyl or dihydroxyalkyl radicals;

R₁₆ is chosen from:

a radical

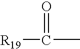

linear or branched, saturated or unsaturated C₁-C₂₂ hydrocarbon-based radicals R₂₀,

a hydrogen atom,

R₁₇ is chosen from:

a radical

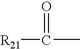

linear or branched, saturated or unsaturated C₁-C₆ hydrocarbon-based radicals R₂₂,

a hydrogen atom,

R₁₇, R₁₉ and R₂₁, which may be identical or different, are chosen from linear or branched, saturated or unsaturated C₇-C₂₁ hydrocarbon-based radicals;

r, n and p, which may be identical or different, are integers ranging from 2 to 6;

y is an integer ranging from 1 to 10;

x and z, which may be identical or different, are integers ranging from 0 to 10;

X⁻ is a simple or complex, organic or inorganic anion; with the proviso that the sum x+y+z is from 1 to 15, that when x is 0, then R₁₆ denotes R₂₀ and that when z is 0, then R,₁₈ denotes R₂₂.

The alkyl radicals R₁₅ may be linear or branched, and in at least one embodiment, the alkyl radicals are linear.

For instance, according to at least one embodiment, R₁₅ is chosen from methyl, ethyl, hydroxyethyl and dihydroxypropyl radicals, and in a further embodiment, from methyl and ethyl radicals.

According to at least one embodiment, the sum x+y+z is from 1 to 10.

When R₁₆ is a hydrocarbon-based radical R₂₀, it may be long and contain from 12 to 22 carbon atoms, or short and contain from 1 to 3 carbon atoms.

When R₁₈ is a hydrocarbon-based radical R₂₂, it may contain 1 to 3 carbon atoms.

For example, according to at least one embodiment, R₁₇, R₁₉ and R₂₁, which may be identical or different, are chosen from linear or branched, saturated or unsaturated C₁₁-C₂₁ hydrocarbon-based radicals, and in a further embodiment, from linear or branched, saturated or unsaturated C₁₁-C₂₁ alkyl and alkenyl radicals.

According to at least one embodiment, x and z, which may be identical or different, are 0 or 1.

According to at least one embodiment, y is equal to 1.

Further, according to at least one embodiment, r, n and p, which may be identical or different, are equal to 2 or 3, for instance equal to 2.

In at least one embodiment, the anion X⁻ may be a halide (chloride, bromide or iodide) or a C₁-C₄ alkyl sulfate, such as methyl sulfate. However, methanesulfonate, phosphate, nitrate, tosylate, an anion derived from an organic acid, such as acetate or lactate, or any other anion that is compatible with the ammonium containing an ester function may also be used.

For example, the anion X⁻ may be chosen from chloride or methyl sulfate.

According to at least one embodiment of the present disclosure, use may be made in the composition of the ammonium salts of formula (IV) in which:

R₁₅ is a methyl or ethyl radical,

x and y are equal to 1;

z is equal to 0 or 1;

r, n and p are equal to 2;

R₁₆ is chosen from:

a radical

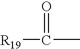

methyl, ethyl or C₁₄-C₂₂ hydrocarbon-based radicals,

a hydrogen atom;

R₁₈ is chosen from:

a radical

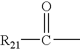

a hydrogen atom;

R₁₇, R₁₉ and R₂₁, which may be identical or different, are chosen from linear or branched, saturated or unsaturated C₁₃-C₁₇ hydrocarbon-based radicals, and further for example from linear or branched, saturated or unsaturated C₁₃-C₁₇ alkyl and alkenyl radicals.

According to at least one embodiment of the present disclosure, the hydrocarbon-based radicals are linear.

Examples of compounds of formula (VIII) that may be mentioned include the salts (such as chloride or methyl sulfate) of diacyloxyethyldimethylammonium, of diacyloxyethylhyd roxyethylmethylammonium, of monoacyloxyethyldihydroxyethyl-methylammonium, of triacyloxyethylmethylammonium, of monoacyloxyethyl-hydroxyethyldimethylammonium, and mixtures thereof. The acyl radicals may contain 14 to 18 carbon atoms and may be derived from a plant oil, for instance palm oil or sunflower oil. When the compound contains several acyl radicals, these radicals may be identical or different.

These products are obtained, for example, by direct esterification of optionally oxyalkylenated triethanolamine, triisopropanolamine, alkyldiethanolamine or alkyldiisopropanolamine onto fatty acids or onto mixtures of fatty acids of plant or animal origin, or by transesterification of the methyl esters thereof. This esterification is followed by a quaternization using an alkylating agent such as an alkyl halide (such as a methyl or ethyl halide), a dialkyl sulfate (such as dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Cognis, Stepanquat® by the company Stepan, Noxamium® by the company Ceca, and Rewoquat® WE 18 by the company Rewo-Goldschmidt.

The composition according to the present disclosure may for instance contain a mixture of quaternary ammonium mono-, di- and triester salts with a weight majority of diester salts.

Examples of mixtures of ammonium salts that may be used include the mixture comprising 15% to 30% by weight of acyloxyethyldihydroxyethylmethylammonium methyl sulfate, from 45% to 60% of diacyloxyethylhydroxylethylmethylammonium methyl sulfate and from 15% to 30% of triacyloxyethylmethylammonium methyl sulfate, the acyl radicals containing from 14 to 18 carbon atoms and being derived from optionally partially hydrogenated palm oil.

It is also possible to use the ammonium salts comprising at least one ester function described in U.S. Pat. Nos. 4,874,554 and 4,137,180.

Among the quaternary ammonium salts mentioned above that may be used for example are those corresponding to formula (V). Non-limiting mention may be made of tetraalkylammonium chlorides, for instance dialkyldimethylammonium or alkyl-trimethylammonium chlorides in which the alkyl radical contains from about 12 to 22 carbon atoms, such as behenyltrimethylammonium, distearyidimethylammonium, cetyltrimethylammonium or benzyldimethylstearylammonium chlorides, or alternatively, secondly, palmitylamidopropyltrimethylammonium chloride or stearamidopropyldimethyl-(myristyl acetate)ammonium chloride corresponding to Quaternium-70 (CTFA 2002) sold under the name Ceraphyl® 70 by the company ISP.

As disclosed herein, the cationic surfactants that may be used include but are not limited to those chosen from quaternary ammonium salts, such as from behenyltrimethylammonium chloride, cetyltrimethylammonium chloride, Quaternium-83;Quaternium-87, behenylamidopropyl-2,3-dihydroxypropyldimethyl-ammonium chloride, palmitylamidopropyltrimethylammonium chloride and stearamidopropylamine.

The composition according to the present disclosure comprises the at least one cationic surfactant in an amount ranging from 0.1% to 10% by weight, for example from 0.5% to 5% by weight relative to the total weight of the composition.

The solid fatty substances according to the present disclosure may be crystalline, amorphous or pasty.

As disclosed herein, the melting point may range from 35 to 250° C., for example from 40 to 150° C.

These solid fatty substances have a viscosity, at a temperature of 40° C. and at a shear rate of 1 s^{-1,} ranging from 1 Pa-s to 1,000,000 Pa-s, for example ranging from 10 to 1,000 Pa-s.

The viscosity measurements may be taken at a temperature of about 40C., using a Carri-Med CSL2-500 viscometer.

The melting points may be measured by DSC or on a Köfler bench. The melting point may be measured by differential calorimetric analysis (DSC) with a temperature increase rate of 10° C./minute. The melting point is then the temperature corresponding to the top of the endothermic melting peak obtained during the measurement.

The non-silicone solid fatty substances with a melting point of greater than or equal to 35° C. may be chosen from oxyethylenated or non-oxyethylenated fatty alcohols, fatty esters, mineral waxes and organic waxes other than fatty esters and fatty alcohols, and mixtures thereof.

For instance, the fatty alcohols according to the present disclosure are linear and saturated, and contain from 12 to 40 carbon atoms.

According to at least one embodiment of the present disclosure, the fatty alcohols have the structure R—OH, in which R is a C₁₂-C₂₄ alkyl group. R may be substituted with at least one hydroxyl group, but in at least one embodiment, R is unsubstituted.

Non-limiting examples that may be mentioned include myristyl alcohol, cetyl alcohol, stearyl alcohol and behenyl alcohol, and mixtures thereof.

The fatty alcohol may be present in a mixture of fatty alcohols, which means that, in a commercial product, several fatty alcohol species may co-exist in the form of a mixture.

In at least one embodiment, the fatty alcohols of the present disclosure are non-oxyalkylenated and/or non-glycerolated. These fatty alcohols may be constituents of animal or plant waxes, for example.

As disclosed herein, the fatty esters include esters containing at least 10 carbon atoms, for example esters of a carboxylic acid containing at least 10 carbon atoms and of a monoalcohol or a polyol. The fatty esters according to the present disclosure may be monoesters, diesters or triesters.

For instance the carboxylic acids may contain from 10 to 30 carbon atoms, for example from 12 to 24 carbon atoms. The alcohols may contain from 10 to 30 carbon atoms, for example from 12 to 24 carbon atoms. As disclosed herein, the solid fatty esters according to the present disclosure include esters of a monocarboxylic fatty acid containing at least 10 carbon atoms and of a monoalcohol containing at least 10 carbon atoms.

Esters according to the present disclosure that may be mentioned include but are not limited to cetyl myristate, myristyl myristate, palmityl palmitate, stearyl palmitate, palmityl stearate and stearyl stearate, and mixtures thereof.

For instance, the fatty esters may be constituents of animal or plant waxes.

For the purposes of the present disclosure, a wax is generally understood to mean a lipophilic compound that is solid at room temperature (about 25° C.), with a reversible solid/liquid change of state, having a melting point of greater than about 40° C., which may be up to 200° C., and having an anisotropic crystal organization in the solid state. In general, the size of the wax crystals is such that the crystals diffract and/or scatter light, giving the composition comprising them a more or less opaque hazy appearance. By bringing the wax to its melting point, it is possible to make it miscible with oils and to form a microscopically uniform mixture, but on returning the temperature of the mixture to room temperature, recrystallization of the wax in the oils of the mixture is obtained, which is detectable microscopically and macroscopically (opalescence).

As waxes that may be used in the present disclosure, non-limiting mention may be made of waxes of animal origin such as beeswax, spermaceti, lanolin wax and lanolin derivatives; plant waxes such as carnauba wax, candelilla wax, ouricury wax, Japan wax, cocoa butter, cork fiber wax or sugarcane wax; mineral waxes, for example paraffin wax, petroleum jelly wax, lignite wax or microcrystalline waxes, ozokerites, olive wax, rice wax, hydrogenated jojoba wax, the absolute waxes of flowers, such as the essential wax of blackcurrant blossom sold by the company Bertin (France), animal waxes, for instance beeswaxes or modified beeswaxes (cerabellina); other waxes or waxy starting materials that may be used according to the present disclosure include, for example, marine waxes such as the product sold by the company Sophim under the reference M82, and mixtures thereof. Organic waxes that may also be mentioned include but are not limited to waxes comprising amide functions, such as natural or synthetic ceramides.

For the definition of waxes, non-limiting mention may be made, for example, of P.D. Dorgan, Drug and Cosmetic Industry, December 1983, pp. 30-33.

In at least one embodiment, the wax(es) is (are) chosen from carnauba wax, candelilla wax, esparto grass wax, paraffin wax, ozokerite, plant waxes, for instance olive wax, rice wax, hydrogenated jojoba wax or the absolute waxes of flowers, such as the essential wax of blackcurrant blossom sold by the company Bertin (France), animal waxes, for instance beeswaxes or modified beeswaxes (cerabellina); other waxes or waxy starting materials that may be used according to the present disclosure include marine waxes, for example, such as the product sold by the company Sophim under the reference M82. The non-silicone solid fatty substance(s) may be presentin the composition in an amount ranging from 0.1% to 10%, for example from 0.5% to 5% and further, for example, from 1% to 4% by weight relative to the total weight of the composition.

The composition according to the present disclosure may optionally contain other surfactants in addition to the above-described compounds of the present disclosure.

The additional surfactants may be present in an amount ranging from about 0.1% to 10%, for example from 0.5% to 8% and even further, for example, from 1% to 5% by weight relative to the total weight of the composition.

In at least one embodiment, the additional surfactants may be chosen from nonionic surfactants.

Nonionic surfactants are compounds that are well known per se (see, e.g.,in this respect “Handbook of Surfactants” by M. R. Porter, published by Blackie & Son (Glasgow and London), 1991, pp. 116-178) and, in the context of the present disclosure, the nature of the non-ionic surfactants is not a critical feature. Thus, they can be chosen from but are not limited to polyethoxylated, polypropoxylated or polyglycerolated fatty alcohols, polyethoxylated, polypropoxylated or polyglycerolated α-diols, polyethoxylated, polypropoxylated or polyglycerolated alkylphenols, and polyethoxylated, polypropoxylated or polyglycerolated fatty acids, all these compounds having a fatty chain containing, for example, 8 to 18 carbon atoms, it being possible for the number of ethylene oxide or propylene oxide groups to range from 2 to 50 and for the number of glycerol groups to range from 2 to 30. Non-limiting mention may also be made of copolymers of ethylene oxide and of propylene oxide, condensates of ethylene oxide and of propylene oxide with fatty alcohols; polyethoxylated fatty amides for instance having from 2 to 30 mol of ethylene oxide, polyglycerolated fatty amides containing on average 1 to 5, for example 1.5 to 4, glycerol groups; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)-alkylamine oxides or N-acylaminopropylmorpholine oxides.

The compositions according to the present disclosure include, in at least one embodiment, non-washing (non-detergent) compositions, and may comprise less than 4% by weight of detergent surfactants, such as anionic surfactants, relative to the total weight of the composition and further, for example, less than 1% by weight. In a further embodiment, the composition as disclosed herein does not contain any detergent surfactants.

The composition according to the present disclosure may also comprise at least one additional conditioning agent. This conditioning agent may be chosen from silicones, cationic polymers, liquid carboxylic fatty esters, plant oils and hydrocarbon-based oils, and mixtures thereof.

The silicones that may be used in accordance with the present disclosure may be soluble or insoluble in the composition, and, for example, they may be polyorgano-siloxanes that are insoluble in the composition of the present disclosure. They may be in the form of oils, waxes, resins or gums. They may be used pure or as an emulsion, a dispersion or a microemulsion.

The organopolysiloxanes are defined in greater detail in Walter Noll's “Chemistry and Technology of Silicones” (1968) Academic Press. They can be volatile or non-volatile.

When they are volatile, the silicones may be chosen from those having a boiling point of between 60° C. and 260° C. For example the silicones may be chosen from:

(i) cyclic silicones containing from 3 to 7, for example 4 to 5, silicon atoms. These are, for example, octamethylcyclotetrasiloxane such as those sold under the name “Volatile Silicone 7207” by Union Carbide or “Silbione 70045 V 2” by Rhodia, decamethylcyclopentasiloxane sold under the name “Volatile Silicone 7158” by Union Carbide, and “Silbione 70045 V 5” by Rhodia, and mixtures thereof.

Non-limiting mention may also be made of cyclocopolymers of the dimethylsiloxane/methylalkylsiloxane type, such as “Silicone Volatile FZ 3109” sold by the company Union Carbide, having the chemical structure:

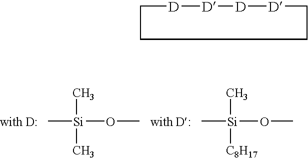

Non-limiting mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is decamethyltetrasiloxane sold under the name “SH 200” by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

The non-volatile silicones that may be mentioned include but are not limited to polyalkylsiloxanes, polyarylsiloxanes, polyalkylarylsiloxanes, silicone gums and resins, polyorganosiloxanes modified with organofunctional groups, and also mixtures thereof.

The organo-modified silicones that can be used in accordance with the present disclosure are silicones as defined above and containing in their structure one or more organo-functional groups attached via a hydrocarbon-based group.

Among the organomodified silicones, non-limiting mention may be made of polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 or the oils Silwet® L 722, L 7500, L 77 and L 711 by the company Union Carbide, and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, for instance, C₁-C₄ aminoalkyl groups;

thiol groups, such as the products sold under the names “GP 72 A” and “GP 71” from Genesee;

alkoxylated groups, such as the product sold under the name “Silicone Copolymer F-755” by SWS Silicones and Abil Wax® 2428, 2434 and 2440 by the company Goldschmidt;

hydroxylated groups, such as the polyorganosiloxanes containing a hydroxyalkyl function, described in French Patent Application FR-A-85/16334;

acyloxyalkyl groups, such as, for example, the polyorganosiloxanes described in U.S. Pat. No. 4,957,732;

anionic groups of the carboxylic acid type, such as, for example, in the products described in European Patent EP 186 507 from the company Chisso Corporation, or of the alkylcarboxylic type, such as those present in the product X-22-3701^{E} from the company Shin-Etsu; 2-hydroxyalkyl sulfonate; 2-hydroxyalkyl thiosulfate such as the products sold by the company Goldschmidt under the names “Abil® S201” and “Abile® S255”;

hydroxyacylamino groups, such as the polyorganosiloxanes described in European Patent Application EP 342 834. Non-limiting mention may be made, for example, of the product Q2-8413 from the company Dow Corning.

Examples of silicones that may be used include but are not limited to polydimethylsiloxanes, polyalkylarylsiloxanes and polydimethylsiloxanes containing amino or alkoxylated groups.

The additional conditioning agents may be present in the composition according to the present disclosure in an amount ranging from 0.01% to 20% by weight, for instance ranging from 0.1% to 10% by weight and further for example ranging from 0.3% to 5% by weight relative to the total weight of the composition.

The cosmetically acceptable medium is aqueous, for instance, and may comprise water or a mixture of water and a cosmetically acceptable solvent such as a C₁-C₄ lower alcohol, for example ethanol, isopropanol, tert-butanol or n-butanol; polyols, for instance propylene glycol or glycerol; polyol ethers; C₅-C₁₀ alkanes; and mixtures thereof.

In at least one embodiment, the solvents may be chosen from glycerol and propylene glycol.

The cosmetically acceptable medium, which is aqueous in at least one embodiment, is present in an amount ranging from 30% to 98% by weight relative to the total weight of the composition.

In at least one embodiment, the solvents are present in concentrations ranging from 0.5% to 30% by weight relative to the total weight of the composition.

The pH of the compositions of the present disclosure is from 2 to 8, and further for example from 3 to 7.

The compositions according to the present disclosure may also contain standard additives that are well known in the art, such as anionic, nonionic or amphoteric polymers, non-polymeric thickeners, for instance acids or electrolytes, opacifiers, nacreous agents, vitamins, provitamins such as panthenol, fragrances, dyes, organic or mineral particles, preserving agents, pH stabilizers, antidandruff agents, for instance piroctone olamine, hair-loss counteractants, grease-control agents and calmatives.

A person skilled in the art will take care to select the optional additives and the amount thereof such that they do not harm the properties of the compositions of the present disclosure.

These additives are present in the composition according to the present disclosure in an amount ranging from 0% to 20% by weight relative to the total weight of the composition.

The compositions of the present disclosure may be in the form of a rinse-out or leave-in hair conditioner, compositions for permanent-waving, relaxing, dyeing or bleaching, or alternatively in the form of rinse-out compositions to be applied before or after a dyeing, bleaching, permanent-waving or relaxing operation or alternatively between the two steps of a permanent-waving or hair-relaxing operation.

They may be used, for example, as hair conditioners, care products, deep-down care masks or scalp treatment lotions or creams. These compositions may be rinse-out or leave-in compositions.

According to at least one embodiment of the present disclosure, the composition may be used as a hair conditioner, for instance, a hair conditioner that can be used on sensitized hair. This hair conditioner may be a rinse-out or leave-in hair conditioner, and in at least one embodiment is a rinse-out hair conditioner.

The cosmetic compositions according to the present disclosure may be in the form of a gel, a milk, a cream, an emulsion, fluid or thickened lotions or a foam, and may be used for the skin, the nails, the eyelashes, the lips, and the hair, for example.

The compositions may be packaged in various forms, for instance in vaporizers, pump-dispenser bottles or in aerosol containers in order to dispense the composition in vaporized form or in the form of a mousse. Such packaging forms are indicated, for example, when it is desired to obtain a spray, a lacquer or a mousse for treating the hair.

The present disclosure also relates to a cosmetic process for treating. keratin materials such as, for example, the skin or the hair, which comprises applying to the keratin materials an effective amount of a cosmetic composition as described above, and optionally rinsing it off after optionally leaving it to act for a period of time.

The rinsing is performed, for example, with water.

Thus, this process according to the present disclosure allows the treatment, conditioning and care of the hair or any other keratin material.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in its respective testing measurement.

The examples that follow are intended to illustrate the present disclosure without, however, being limiting in nature.

### EXAMPLES

### Examples 1 to 4

The following rinse-out hair-conditioning compositions were prepared:
1. All water was introduced into the manufacturing tank, and the water-soluble compounds were then added, with the exception of the oxyethylenated sorbitan ester(s). The mixture was heated to 80° C. with stirring using a doctor blade, until fully dissolved.
2. The water-insoluble compounds were introduced apart from the fragrances and the silicones into an additional tank. The mixture was heated to 80° C.
3. The contents of the additional tank and the silicones, if any, were introduced into the manufacturing tank and emulsified for 10 minutes with a vigorous turbo mixer and doctor blade stirring, while the temperature was maintained. Then, the mixture was cooled.
4. At 30° C., the oxyethylenated sorbitan ester and the fragrances were introduced with doctor blade stirring.

### Example 1

| | In g AM | |
|---|---|---|
| Cetyl alcohol | 2.5 | g |
| Cetyl esters: myristyl/cetyl/stearyl myristate/ | 0.25 | g |
| palmitate/stearate mixture (MIRACETI from | | |
| Laserson) | | |
| Cetyltrimethylammonium chloride at 25% AM | 0.45 | g |
| (ARQUAD 16-25 Io from Akzo Nobel) | | |
| Stearylamidopropyldimethylamine (MACKINE 301 from | 0.75 | g |
| MacIntyre) | | |
| Sorbitan monolaurate oxyethylenated with 4 mol of | 4 | g |
| ethylene oxide (TWEEN 21 from Uniqema) | | |
| Polydimethylsiloxane containing aminoethyl | 0.63 | g |
| iminopropyl end groups, at 35% active material | | |
| (Dow Corning 939 Emulsion from Dow Corning) | | |

| | |
|---|---|
| Fragrance | qs |
| Preserving agents | qs |
| Water | qs 100 |

### Example 2

| | In g AM | |
|---|---|---|
| Cetyl alcohol | 2.5 | g |
| Cetyl esters (MIRACETI from Laserson) | 0.25 | g |
| Cetyltrimethylammonium chloride at 25% AM (ARQUAD | 0.45 | g |
| 16-25 Io from Akzo Nobel) | | |
| Stearylamidopropyldimethylamine (MACKINE 301 from | 0.75 | g |
| MacIntyre) | | |
| Sorbitan monolaurate oxyethylenated with 4 mol of | 4 | g |
| ethylene oxide (TWEEN 21 from Uniqema) | | |
| Polydimethylsiloxane containing aminoethyl iminopropyl | 0.63 | g |
| end groups, at 35% active material (Dow Corning 939 | | |
| Emulsion from Dow Corning) | | |
| Kaolinite | 3 | g |

| | |
|---|---|
| Fragrance | qs |
| Preserving agents | qs |
| Water | qs 100 |

### Example 3

| | In g AM | |
|---|---|---|
| Cetyl alcohol | 5 | g |
| Cetyl esters (MIRACETI from Laserson) | 1 | g |
| Behenyltrimethylammonium chloride at 80% active material | 3.2 | g AM |
| in a water/propanol mixture (GENAMIN KDMP from | | |
| Clariant) | | |
| Sorbitan monolaurate oxyethylenated with 4 mol of | 4 | g |
| ethylene oxide (TWEEN 21 from Uniqema) | | |
| Polydimethylsiloxane containing aminoethyl iminobutyl | 1.8 | g AM |
| end groups, at 60% active material (DC2-8299 from | | |
| Dow Corning) | | |
| Piroctone olamine (OCTOPIROX from Clariant) | 0.5 | g |

| | |
|---|---|
| Fragrance | qs |
| Preserving agents | qs |
| Water | qs 100 |

### Example 4

| | In g AM | |
|---|---|---|
| Cetyl alcohol | 5 | g |
| Behenyltrimethylammonium chloride at 80% active | 3.2 | g AM |
| material in a water/propanol mixture (GENAMIN | | |
| KDMP from Clariant) | | |
| Sorbitan monolaurate oxyethylenated with 20 mol | 4 | g |
| of ethylene oxide (TWEEN 20 from Uniqema) | | |

| | |
|---|---|
| Fragrance | qs |
| Preserving agents | qs |
| Water | qs 100 |

These compositions were applied to washed and wrung hair. After an action time of 2 to 3 minutes, the hair was rinsed with water. No discomfort was reported during or after the action time.

After drying, the hair was easily disentangled, and was soft and shiny.

What is claimed is:

1. A cosmetic composition comprising, in a cosmetically acceptable aqueous medium:
at least one cationic surfactant;
at least one ester of sorbitan and of a saturated or unsaturated, linear or branched C₈-C₃₀ fatty acid with a number of moles of ethylene oxide of less than or equal to 20; and
at least one non-silicone solid fatty substance,
wherein the at least one oxyethylenated sorbitan ester and the at least one fatty substance weight are present in a ratio ranging from 0.2 to 50.

2. The composition according to claim 1, wherein the at least one cationic surfactant is chosen from optionally polyoxyalkylenated primary, secondary ortertiary fatty amine salts and quaternary ammonium salts, and mixtures thereof.

3. The composition according to claim 2, wherein the quaternary ammonium salts are chosen from:
those having formula (V) below: 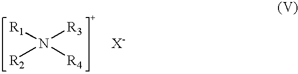
in which
R₁ to R₄, which may be identical or different, are chosen from linear or branched aliphatic radicals containing from 1 to 30 carbon atoms and from aromatic radicals; and
X⁻ is an anion chosen from halides, phosphates, acetates, lactates, (C₂-C₆)alkyl sulfates, alkylsulfonates and alkylarylsulfonates;
quaternary ammonium salts of imidazoline;
diquaternary ammonium salts of formula (VII): 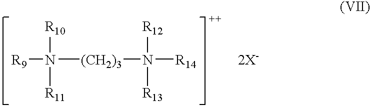
in which
R₉ is an aliphatic radical having from 16 to 30 carbon atoms,
R₁₀, R₁₁, R₁₂, R₁₃ and R₁₄, which may be identical or different, are chosen from hydrogen and alkyl radicals having from 1 to 4 carbon atoms, and
X⁻ is an anion chosen from halides, acetates, phosphates, nitrates and methyl sulfates;
quaternary ammonium salts comprising at least one ester function.

4. The composition according to claim 3, wherein the quaternary ammonium salts of imidazoline are chosen from those of formula (VI) below: 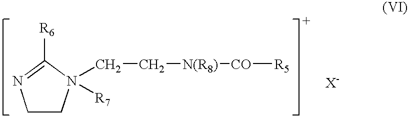
in which
R₅ is chosen from a C₈-C₃₀ alkenyl radical and a C₈-C₃₀ alkyl radical,
R₆ is chosen from a hydrogen atom, a C₁-C₄ alkyl radical, a C₈-C₃₀ alkenyl radical, and a C₈-C₃₀ alkyl radical,
R₇ is a C₁-C₄ alkyl radical,
R₈ is chosen from a hydrogen atom and a C₁-C₄ alkyl radical, and
X⁻ is an anion chosen from halides, phosphates, acetates, lactates, alkyl sulfates, alkylsulfonates and alkylarylsulfonates.

5. The composition according to claim 4, wherein R₅ and R₆ are a mixture of alkenyl or alkyl radicals containing from 12 to 21 carbon atoms, R₇ is methyl, and R₈ is hydrogen.

6. The composition according to claim 1, wherein the at least one cationic surfactant is chosen from behenyltrimethylammonium chloride, cetyltrimethylammonium chloride, Quaternium-83,
Quaternium-87, behenylamidopropyl-2,3-dihydroxypropyldimethylammonium chloride and palm itylamidopropyltrimethylammonium chloride.

7. The composition according to claim 1, wherein the at least one cationic surfactant is present in an amount ranging from 0.05% to 10% by weight relative to the total weight of the composition.

8. The composition according to claim 7, wherein the at least one cationic surfactant is present in an amount ranging from 0.1% to 8% by weight relative to the total weight of the composition.

9. The composition according to claim 1, wherein the fatty acid comprises from 8 to 24 carbon atoms.

10. The composition according to claim 9, wherein the fatty acid comprises from 8 to 18 carbon atoms.

11. The composition according to claim 9, wherein the fatty acid is chosen from lauric acid and stearic acid.

12. The composition according to claim 1 1, wherein the fatty acid is lauric acid.

13. The composition according to claim 1, wherein the oxyethylenated sorbitan esters are monoesters of a C₈-C₂₄ fatty acid and of oxyethylenated sorbitan.

14. The composition according to claim 1, wherein the number of moles of ethylene oxide is less than 10 mol.

15. The composition according to claim 14, wherein the number of moles of ethylene oxide is equal to 4 mol.

16. The composition according to claim 1, wherein the oxyethylenated sorbitan esters are chosen from sorbitan monolaurate oxyethylenated with 4 mol of ethylene oxide (4 EO) and sorbitan monostearate oxyethylenated with 4 mol of ethylene oxide (4 EO).

17. The composition according to claim 1, wherein the oxyethylenated sorbitan ester is sorbitan monolaurate oxyethylenated with 4 mol of ethylene oxide (4 EO).

18. The composition according to claim 1, wherein the oxyethylenated sorbitan ester is present in the composition in an amount ranging from 0.4% to 10% by weight relative to the total weight of the said composition.

19. The composition according to claim 18, wherein the oxyethylenated sorbitan ester is present in the composition in an amount ranging from 0.4% to 8% by weight relative to the total weight of the said composition.

20. The composition according to claim 1, wherein the at least one non-silicone solid fatty substance is chosen from oxyethylenated or non-oxyethylenated fatty alcohols, fatty esters, mineral waxes, organic waxes other than fatty esters and fatty alcohols, and mixtures thereof.

21. The composition according to claim 1, wherein the at least one non-silicone solid fatty substance is chosen from solid fatty alcohols and solid fatty esters.

22. The composition according to claim 1, wherein the at least one non-silicone solid fatty substance is a linear fatty alcohol.

23. The composition according to claim 1, wherein the at least one non-silicone solid fatty substance is a saturated fatty alcohol.

24. The composition according to claim 1, wherein the at least one non-silicone solid fatty substance is a fatty alcohol having the structure R—OH, in which R is an optionally hydroxylated alkyl radical having from 12 to 24 carbon atoms.

25. The composition according to claim 1, wherein the at least one non-silicone solid fatty substance is a fatty alcohol chosen from myristyl alcohol, cetyl alcohol, behenyl alcohol and stearyl alcohol, and mixtures thereof.

26. The composition according to claim 20, wherein the fatty esters are esters of a carboxylic acid and of a monoalcohol or a polyol, the ester having at least 10 carbon atoms.

27. The composition according to claim 25, wherein the fatty esters are chosen from esters of a monocarboxylic fatty acid having at least 10 carbon atoms and of a monoalcohol having at least 10 carbon atoms.

28. The composition according to claim 27, wherein the fatty esters are chosen from cetyl myristate, myristyl myristate, palmityl palmitate, stearyl palmitate, palmityl stearate and stearyl stearate, and mixtures thereof.

29. The composition according to claim 20, wherein the waxes are chosen from beeswax, spermaceti, lanolin wax and lanolin derivatives; carnauba wax, candelilla wax, ouricury wax, Japan wax, cocoa butter, cork fiber wax or sugarcane wax; paraffin wax, petroleum jelly wax, lignite wax or microcrystalline waxes, ozokerites, olive wax, rice wax, hydrogenated jojoba wax, the absolute waxes of flowers, beeswaxes or modified beeswaxes (cerabellina), and natural or synthetic ceramides, and mixtures thereof.

30. The composition according to claim 1, wherein the non-silicone solid fatty substance is present in the composition in an amount ranging from 0.1% to 10% by weight relative to the total weight of the composition.

31. The composition according to claim 30, wherein the non-silicone solid fatty substance is present in the composition in an amount ranging from 0.5% to 5% by weight relative to the total weight of the composition.

32. The composition according to claim 1, wherein the oxyethylenated sorbitan ester/non-silicone solid fatty substance ratio ranges from 0.25 to 10.

33. The composition according to claim 32, wherein the oxyethylenated sorbitan ester/non-silicone solid fatty substance ratio ranges from 0.4 to 2.

34. The composition according to claim 1, wherein it also comprises at least one additional conditioning agent.

35. The composition according to claim 34, wherein the at least one additional conditioning agent is chosen from silicones, cationic polymers, liquid carboxylic fatty esters, plant oils and hydrocarbon-based oils, and mixtures thereof.

36. The composition according to claim 1, wherein the cosmetically acceptable aqueous medium comprises water or a mixture of water and of a cosmetically acceptable solvent.

37. The composition according to claim 36, wherein the cosmetically acceptable solvent is chosen from C₁-C₄ lower alcohols; alkylene glycols; polyol ethers; C₅-C₁₀ alkanes, and mixtures thereof.

38. The composition according to claim 1, further comprising at least one additive chosen from anionic, nonionic or amphoteric polymers, thickeners, opacifiers, nacreous agents, vitamins, provitamins, fragrances, dyes, organic or mineral particles, preserving agents and pH stabilizers.

39. The composition according to claim 1, wherein the composition is in the form of a hair conditioner, a composition for permanent-waving, relaxing, dyeing or bleaching the hair, or a rinse-out composition to be applied between the two steps of a permanent-waving or hair-relaxing operation.

40. The composition according to claim 1, wherein the composition is in the form of a rinse-out hair conditioner.

41. A cosmetic process for treating keratin materials, comprising:
applying to the keratin materials an effective amount of a cosmetic composition;
wherein the cosmetic composition comprises, in a cosmetically acceptable aqueous medium:
at least one cationic surfactant,
at least one ester of sorbitan and of a saturated or unsaturated, linear or branched C₈-C₃₀ fatty acid with a number of moles of ethylene oxide of less than or equal to 20, and
at least one non-silicone solid fatty substance, in an oxyethylenated sorbitan ester/fatty substance weight ratio ranging from 0.2 to 50;
and optionally rinsing off the cosmetic composition, after optionally leaving the cosmetic composition to act for a period of time.
